(19) United States
(12) Patent Application Publication
(10) Pub. No.: US 2020/0203003 A1
(43) Pub. Date: Jun. 25, 2020

(54) MANAGEMENT DEVICE AND MANAGEMENT SYSTEM

(71) Applicant: CANON MEDICAL SYSTEMS CORPORATION, Otawara-shi (JP)

(72) Inventors: Yosuke Yanagida, Nasushiobara (JP); Kenji Matsue, Nasushiobara (JP); Maiko Tezuka, Nasushiobara (JP); Yosuke Okubo, Nasushiobara (JP)

(73) Assignee: CANON MEDICAL SYSTEMS CORPORATION, Otawara-shi (JP)

(21) Appl. No.: 16/720,250

(22) Filed: Dec. 19, 2019

(30) Foreign Application Priority Data
Dec. 19, 2018 (JP) 2018-236942

(51) Int. Cl. G16H 30/40; G16H 15/00; G06K 9/00

(52) U.S. Cl. CPC G16H 30/40; G06K 9/00536; G06K 9/00523; G16H 15/00

(57) ABSTRACT

A management device in an embodiment includes a processing circuit configured to collect identification information that identifies an application utilized for image analysis of a medical image utilized for generation of a medical report from a plurality of medical reports, and to output information indicating utilization status of the application by using a plurality of pieces of the collected identification information.

### CROSS-REFERENCE TO RELATED APPLICATIONS

This application is based upon and claims the benefit of priority from Japanese Patent Application No. 2018-236942, filed on Dec. 19, 2018; the entire contents of which are incorporated herein by reference.

### FIELD

An embodiment described herein relates generally to a management device and a management system.

### BACKGROUND

At hospitals or the like, there has been a demand for grasping a utilization result of applications utilized for image analysis of medical images. To meet this demand, a technology is known by which a service center who provides dedicated applications for medical image processing systems corresponding to the purposes of diagnosis and treatment calculates fees based on the utilization result of the dedicated applications. Furthermore, a technology is known in which a workflow manager selects an appropriate application for generating medical information that a user needs and in which information relating to the selected application is stored.

### BRIEF DESCRIPTION OF THE DRAWINGS

FIG. 1 is a diagram illustrating one example of a configuration of a result management system according to a first embodiment;

FIG. 2 is a diagram illustrating one example of functional blocks of the result management system in the first embodiment;

FIG. 3 is a diagram illustrating one example of an application table in the first embodiment;

FIG. 4 is a diagram illustrating one example of an order table in the first embodiment;

FIG. 5 is a diagram illustrating one example of a report table in the first embodiment;

FIG. 6 is a diagram illustrating one example of a result display screen by date in the first embodiment;

FIG. 7 is a diagram illustrating one example of a result display screen by each clinical application in the first embodiment;

FIG. 8 is a diagram illustrating one example of a result display screen of an individual clinical application in the first embodiment;

FIG. 9 is a diagram illustrating one example of a result display screen by each assumed disease name in the first embodiment;

FIG. 10 is a sequence diagram illustrating one example of a flow of a result management process in the first embodiment; and

FIG. 11 is a flowchart illustrating one example of a purpose determination process in the first embodiment.

### DETAILED DESCRIPTION

A management device in an embodiment includes a processing circuit configured to collect identification information that identifies an application utilized for image analysis of a medical image utilized for generation of a medical report from a plurality of medical reports, and to output information indicating utilization status of the application by using a plurality of pieces of the collected identification information.

With reference to the accompanying drawings, the following describes an exemplary embodiment of a management device and a management system in detail. Possible embodiments are not limited to the embodiments described below. Further, the description of each of the embodiments is, in principle, similarly applicable to any other embodiment.

In the present embodiment, a medical image represents an image of a patient captured by a medical diagnostic imaging apparatus (modality) such as an X-ray Computed Tomography (CT) device, a Magnetic Resonance Imaging (MRI) device, an ultrasonic diagnostic apparatus, and the like, for example. In the present embodiment, image analysis represents performing image processing on a medical image for the purposes of assisting image interpretation and check by a doctor who is responsible for image interpretation on the medical image in a clinical use (hereinafter may also be referred to as “image interpreter”), or by an engineer who checks the image. Examples of the image analysis in the present embodiment include, but not limited to, a process of removing bone portions from the medical image or a process of extracting blood vessel portions, a volume rendering (VR) process of generating two-dimensional image data on which three-dimensional information is reflected, and the like. In the following description, an application utilized for image analysis of a medical image may be referred to as “clinical application”.

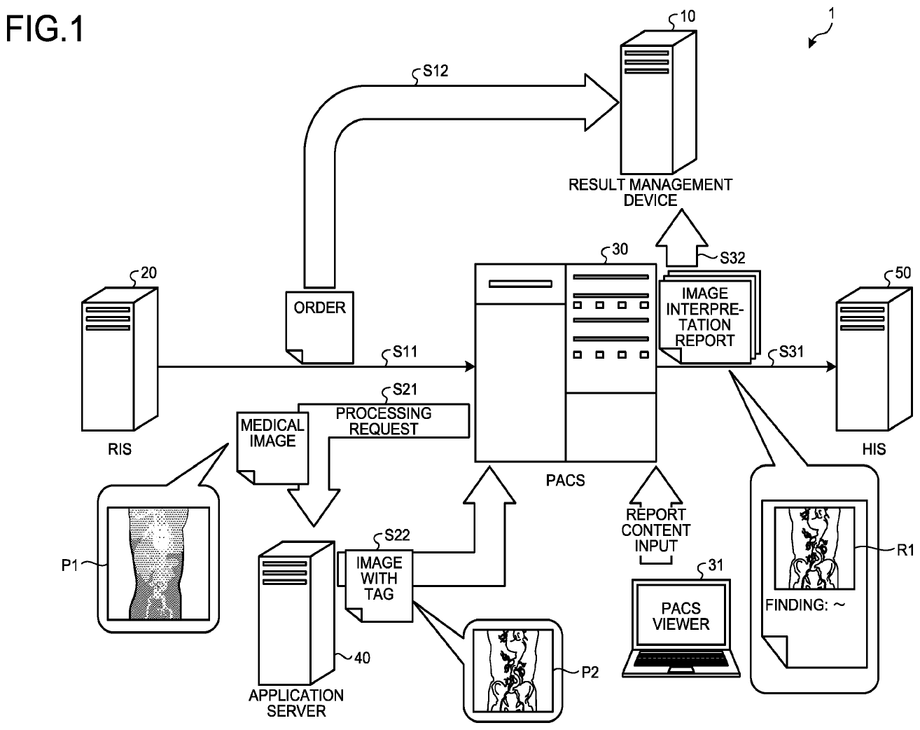

FIG. 1 is a diagram illustrating one example of a configuration of a result management system according to a first embodiment. As illustrated in FIG. 1, a result management system 1 according to the present embodiment includes a result management device 10, a Radiology Information System (RIS) 20, a Picture Archiving and Communication System (PACS) 30, a PACS viewer 31, an application server 40, and a Hospital Information System (HIS) 50.

In FIG. 1, the result management device 10 collects information about a clinical application utilized for image analysis on a medical image, and outputs information on utilization status of the clinical application in the image analysis, for example. The RIS 20 outputs an order for capturing a medical image by various types of modalities and an order for a medical report based on the medical image. The RIS 20 is implemented by a worklist server conforming to a Digital Imaging and Communications in Medicine (DICOM) specification, for example.

In FIG. 1, the PACS 30 is a device that stores therein the collected medical images and performs various image processing on the medical images. The PACS 30 may use an external clinical application when performing various image processing. The PACS viewer 31 is a terminal that receives operation on the PACS 30, and displays the information that is output from the PACS 30, for example. The PACS viewer 31 is operated by a manager (not depicted) of the PACS 30, an image interpreter (not depicted), and the like, for example.

In FIG. 1, the application server 40 is a computer that executes a clinical application, for example. The application server 40 performs, by executing a clinical application under the instruction by the PACS 30, image analysis on the medical image and outputs an execution result to the PACS 30. The HIS 50 is a system that manages information on the entire hospital such as generation of an electronic medical record, management of medical accounting, and the like. In the following description, because various devices other than the result management device 10 are implemented by a known computer, detailed description thereof is omitted.

In FIG. 1, the RIS 20 first outputs an order such as generation of a medical report using a medical image to the PACS 30 (Step S11). In the present embodiment, the RIS 20 outputs an order for the generation of an image interpretation report on the medical image. At this time, the result management device 10 acquires information on the order that is output from the RIS 20 (Step S12). In the present embodiment, an order may further request for capturing a medical image, and an order for capturing the medical image may further include an order for the generation of an image interpretation report.

The PACS 30 requests, upon receiving a request for image analysis utilizing a clinical application from the PACS viewer 31 that is operated by an image interpreter for example, the image analysis on a medical image P1 from the application server 40 that executes the relevant clinical application (Step S21).

The application server 40 performs, upon receiving from the PACS 30 the request for image analysis, the image analysis on the medical image P1 and generates a new medical image P2. The new medical image P2 is an image generated by the clinical application performing image analysis for vessel identification on the medical image P1, for example. The application server 40 further attaches a tag indicating information identifying the clinical application that executed the image analysis to the new medical image P2 and outputs the tagged medical image P2 to the PACS 30 (Step S22).

The PACS 30 requests the PACS viewer 31 to display the medical image P2, for example. The PACS viewer 31 receives, for example, a text input of an image interpretation report on the medical image P2 from the image interpreter who referred to the medical image P2. Then, the PACS 30 outputs an image interpretation report R1 including the medical image P2 and the input text received from the image interpreter to the HIS 50 (Step S31). At that time, the result management device 10 acquires information on the image interpretation report R1 that is output from the PACS 30 (Step S32). The result management device 10, by using the information on the acquired image interpretation report R1 and the information on the order corresponding to the image interpretation report R1, summarizes and outputs the utilization status of the clinical application that was utilized in generating the image interpretation report.

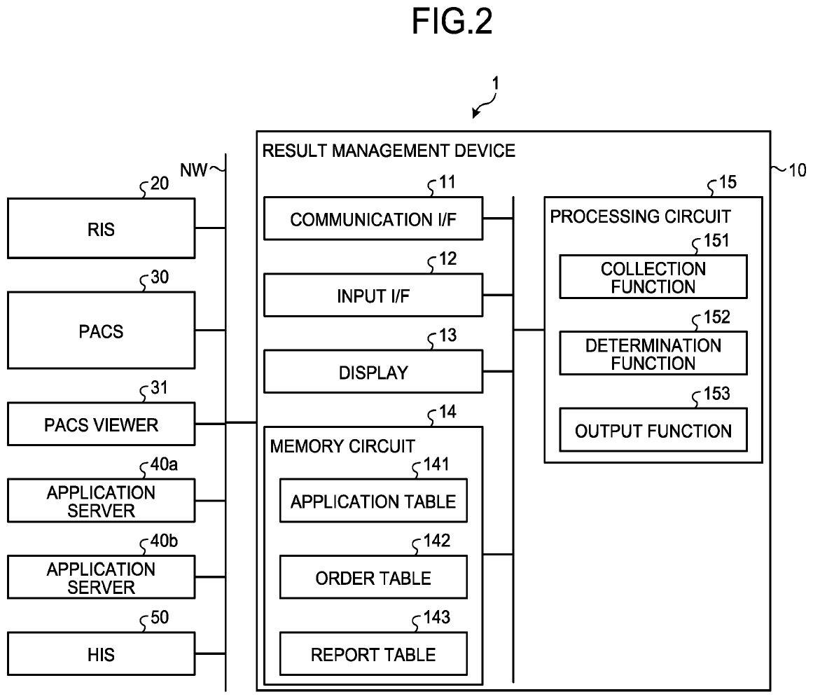

FIG. 2 is a diagram illustrating one example of functional blocks of the result management system in the first embodiment. As illustrated in FIG. 2, in the result management system 1, the result management device 10, the RIS 20, the PACS 30, the PACS viewer 31, application servers 40a and 40b, and the HIS 50 are connected to one another via a network NW that is either wired or wireless in a hospital. On the network NW, various other devices (not depicted) in the hospital such as modality are also connected. The number of application servers 40 included in the result management system 1 is arbitrary, and it may be a configuration in which only one application server 40 is included or may be a configuration in which the application servers 40 of three or more are included. In the following description, the application servers 40a and 40b may simply be described as “application server 40” when presented without distinction.

In FIG. 2, the result management device 10 includes a communication interface (I/F) 11, an input I/F 12, a display 13, a memory circuit 14, and a processing circuit 15.

The communication I/F 11 is coupled to the processing circuit 15 and controls the transmission of various data and the communication that are performed with the result management device 10 connected via the network NW. For example, the communication I/F 11 may be realized by using a network card, a network adaptor, a Network interface Controller (NIC), or the like.

The input I/F 12 is coupled to the processing circuit 15, converts the input operation received from a manager (not depicted) of the result management device 10 into an electrical signal, and outputs the converted electrical signal to the processing circuit 15. For example, the input I/F 12 may be a switch button, a mouse, a keyboard, a touch panel, and/or the like.

The display 13 is coupled to the processing circuit 15 and displays various information and various image data that are output from the processing circuit 15. For example, the display 13 may be realized by using a liquid crystal monitor, a Cathode Ray Tube (CRT) monitor, a touch panel, or the like.

The memory circuit 14 is coupled to the processing circuit 15 and stores therein various data. For example, the storage 14 may be realized by using a semiconductor memory element such as a Random Access Memory (RAM) or a flash memory, or a hard disk, an optical disk, or the like. For example, in the present embodiment, as illustrated in FIG. 2, the memory circuit 14 stores therein an application table 141, an order table 142, and a report table 143.

The application table 141 stores therein information about the utilization purpose of clinical applications and the like. The information stored in the application table 141 is input by the application server 40 or is input by the manager of the result management device 10, for example.

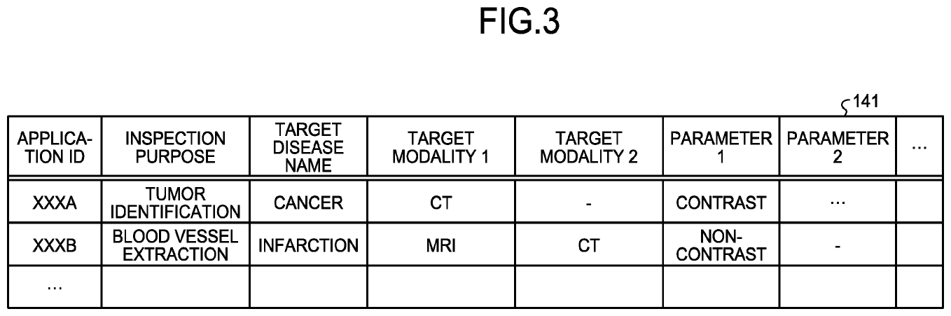

FIG. 3 is a diagram illustrating one example of the application table in the first embodiment. For example, the application table 141 is, as illustrated in FIG. 3, the information that associates “application ID” with “inspection purpose”, “target disease name”, “target modality 1”, “target modality 2”, “parameter 1”, and “parameter 2”. In FIG. 3, “application ID” presents an identifier that uniquely identifies the relevant clinical application. “Inspection purpose” presents the purpose of image analysis by the relevant clinical application. “Target disease name” presents the name of disease to be a target of image analysis of the relevant clinical application. “Target modality 1” and “target modality 2” present modality by which the medical image to be the object of image analysis by the relevant clinical application has been captured. “Parameter 1” and “parameter 2” present attribute of the medical image for which the relevant clinical application is suitable. In the table illustrated in FIG. 3, although “target modality” and “parameter” are provided with two fields each, the number of fields is not limited thereto. In addition, other information such as an inspection target portion and the like may further be included. In the following description, a field where no data is registered is indicated by “−”. The same applies to the other tables in the following description.

For example, the first record of the table illustrated in FIG. 3 indicates that the clinical application of the application ID “XXXA” is intended for “tumor identification” for “cancer” and is targeting the medical image captured in a “contrast” manner by “CT”. The second record of the table illustrated in FIG. 3 indicates that the clinical application of the application ID “XXXB” is intended for “blood vessel extraction” for “infarction” and is targeting the medical image captured in a “non-contrast” manner by “CT” or “MRI”.

Next, the order table 142 stores therein information about an order for generating image interpretation report that is output from the RIS, for example. The order table 142 is set by a collection function 151 which will be described later, for example.

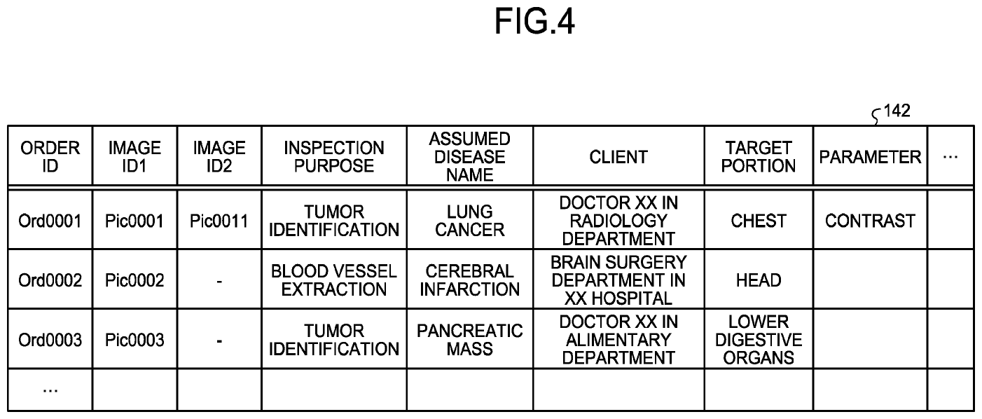

FIG. 4 is a diagram illustrating one example of the order table in the first embodiment. For example, the order table 142 is, as illustrated in FIG. 4, the information that associates “order ID” with “image ID1”, “image ID2”, “inspection purpose”, “assumed disease name”, “client”, “target portion”, and “parameter”. In FIG. 4, “order ID” presents an identifier that uniquely identifies the relevant order. “Image ID1” and “image ID2” present identifier that uniquely identifies an image to be an object of image interpretation in the relevant order. “Inspection purpose” presents a inspection purpose of the image interpretation report to be an object of the relevant order. “Assumed disease name” presents the name of disease that is included in the relevant order and assumed at the time of outputting the order. “Client” presents a doctor, hospital, and the like who output the relevant order. “Target portion” presents the portion to be a target of image interpretation in the relevant order. “Parameter” presents an attribute of the medical image to be an object of the relevant image interpretation. In the table illustrated in FIG. 4, two fields of “image ID” are provided and one field of “parameter” is provided, but the number of fields is not limited thereto. In addition, other information, for example, an order type such as representing whether the order is related to an order for capturing a medical image or an order for the generation of a report, a type and device of modality, a protocol, an imaging method, the name of an engineer who performs image capturing, and the like may further be included.

For example, the first record of the table illustrated in FIG. 4 indicates that the order of the order ID “Ord0001” is requesting image interpretation on the medical images of “chest” captured in a “contrast” manner having the image IDs “Pic0001” and “Pic0011” for the purpose of “tumor identification” that assumed “lung cancer”, and was requested by “doctor xx in radiology department”. Furthermore, for example, the second record of the table illustrated in FIG. 4 indicates that the order of the order ID “Ord0002” is requesting image interpretation on the medical image of “head” having the image ID “Pic0002” for the purpose of “blood vessel extraction” that assumed “cerebral infarction”, and was requested by “brain surgery department in xx hospital”. For example, the third record of the table illustrated in FIG. 4 indicates that the order of the order ID “Ord0003” is requesting image interpretation on the medical image of “lower digestive organs” having the image ID “Pic0003” for the purpose of “tumor identification” that assumed “pancreatic mass”, and was requested by “doctor xx in alimentary department”.

Next, the report table 143 stores therein information about the image interpretation report generated in accordance with the order. The report table 143 is set by the collection function 151, for example.

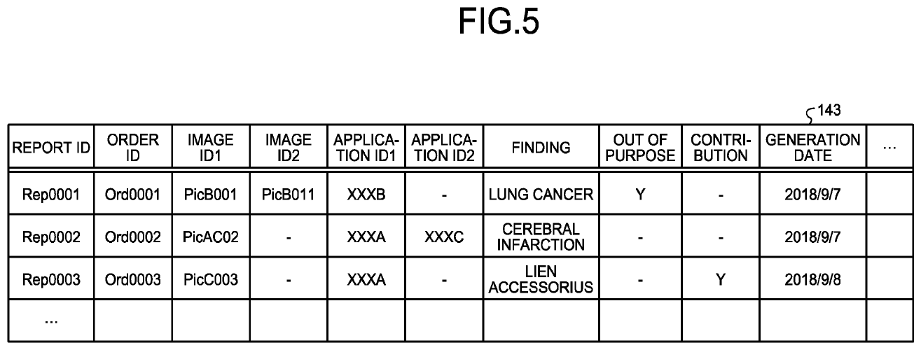

FIG. 5 is a diagram illustrating one example of the report table in the first embodiment. For example, the report table 143 is, as illustrated in FIG. 5, the information associating “report ID” with “order ID”, “image ID1”, “image ID2”, “application ID1”, “application ID2”, “finding”, “out of purpose”, “contribution”, and “generation date”. In FIG. 5, “report ID” presents an identifier that uniquely identifies the image interpretation report generated in accordance with the order ID. “Image ID1” and “image ID2” store therein the image ID that uniquely identifies the image on which image analysis was performed by a clinical application, which was the object of the relevant image interpretation report. “Application ID1” and “application ID2” present a tag that identifies the clinical application utilized for the image analysis of either of the images of “image ID1” and “image ID2”. In the present embodiment, the tag that identifies the clinical application is the application ID of the relevant clinical application that is attached to the medical image that was the object of the image analysis, for example. “Finding” presents the name of disease presented in the relevant image interpretation report. “Out of purpose” is a flag indicating that the clinical application utilized for image analysis in generating the relevant image interpretation report was utilized for the purpose other than the inspection purpose of the relevant clinical application. “Contribution” is a flag indicating that the clinical application utilized for image analysis in generating the relevant image interpretation report contributed to determine the finding in the relevant image interpretation report. In each flag, “Y” is registered when the condition is met and, when the condition is not met, “−” is given. “Generation date” presents the date on which the relevant image interpretation report was generated. The number of fields of “application ID1” and “application ID2” is not limited to two. In generating the image interpretation report, when the clinical application was not utilized, the fields of “application ID1” and “application ID2” are “−”.

For example, the first record of the table illustrated in FIG. 5 stores therein the fact that the image interpretation report of the report ID “Rep0001” in accordance with the order of the order ID “Ord0001” is about the images of the image IDs “PicB001” and “PicB011” on which image analysis was performed by the clinical application of the application ID “XXXB” and that the finding is “lung cancer”. Furthermore, the first record of the table illustrated in FIG. 5 stores therein the fact that the relevant image interpretation report was generated on “Sep. 7, 2018”, and that the clinical application of the application ID “XXXB” was utilized for a purpose other than the inspection purpose of the relevant clinical application, and did not contribute to the finding. In the present embodiment, the image of the image ID “PicB001” is an image generated by the image analysis performed on the image of the image ID “Pic0001” by the clinical application of the application ID “XXXB”, and the image of the image ID “PicB011” is an image generated by the image analysis performed on the image of the image ID “Pic0011” by the clinical application of the application ID “XXXB”. Furthermore, for example, the second record of the table illustrated in FIG. 5 stores therein the fact that the image interpretation report of the report ID “Rep0002” in accordance with the order of the order ID “Ord0002” is about the image of the image ID “PicAC02” on which image analysis was performed by the clinical application of the application ID “XXXA” and by the clinical application of the application ID “XXXC” and that the finding is “cerebral infarction”. The second record of the table illustrated in FIG. 5 stores therein the fact that the relevant image interpretation report was generated on “Sep. 7, 2018”, and that the image analysis utilizing the clinical applications of the application IDs “XXXA” and “XXXC” neither corresponds to the purpose other than the inspection purpose of the relevant clinical application, nor contributed to the finding. Moreover, for example, the third record of the table illustrated in FIG. 5 stores therein the fact that the image interpretation report of the report ID “Rep0003” in accordance with the order of the order ID “Ord0003” is about the image of the image ID “PicC003” on which image analysis was performed by the clinical application of the application ID “XXXA” and that the finding is “lien accessorius”. The third record of the table illustrated in FIG. 5 stores therein the fact that the relevant image interpretation report was generated on “Sep. 8, 2018”, and that the image analysis utilizing the clinical application of the application ID “XXXA” does not correspond to the utilization for out of purpose, and contributed to the finding.

Referring back to FIG. 2, the processing circuit 15 executes the collection function 151, a determination function 152, and an output function 153. The collection function 151 is one example of a collection unit. The determination function 152 is one example of a determination unit. The output function 153 is one example of an output unit. In this situation, for example, processing functions of the constituent elements of the processing circuit 15 illustrated in FIG. 2, namely, the correction function 151, the determination function 152 and the output function 153, are recorded in the storage 14 in the form of computer-executable programs. The processing circuit 15 is configured to realize the functions corresponding to the programs, by reading the programs from the storage 14 and executing the read programs. In other words, the processing circuit 15 that has read the programs has the functions illustrated within the processing circuit 15 in FIG. 2.

In this situation, all the processing functions of the correction function 151, the determination function 152, and the output function 153 may be recorded in the storage 14 in the form of one computer-executable program. For example, the program may be referred to as a medical information management program. In that situation, the processing circuit 15 realizes the correction function 151, the determination function 152, and the output function 153 corresponding to the medical information management program, by reading the medical information management program from the storage 14 and executing the read medical information management program.

The collection function 151 in the processing circuit 15 collects various kinds of information on the clinical application, order, and image interpretation report. For example, the collection function 151 collects, from the application server 40, the information about the application ID on the introduced clinical application, inspection purpose, target disease name, and the like and stores the collected information in the application table 141. Alternatively, the collection function 151 collects, from the manager of the result management device 10, the information about the application ID on the clinical application, inspection purpose, target disease name, and the like that was input via the input I/F 12 and stores the collected information in the application table 141. In addition, when the RIS 20 transmits an order that requests an image interpretation report to the PACS 30 for example, the collection function 151 collects information on the order ID, image ID, inspection purpose, assumed disease name, and the like about the relevant order and stores the collected information in the order table 142. In other words, the record in the order table 142 is increased one by one each time the collection function 151 collects the information on the order. In addition, when the PACS 30 transmits the image interpretation report to the HIS 50 for example, the collection function 151 collects information including the report ID, image ID, finding, and the like about the relevant image interpretation report and stores the collected information in the report table 143. At this time, the collection function 151 accesses the PACS 30 and, when the tag information has been attached to the image corresponding to the image ID, the collection function 151 acquires the relevant tag information, and stores the collected information in the report table 143, for example. In other words, the record in the report table 143 is increased one by one each time the collection function 151 collects the information on the order.

The collection function 151 acquires a tag included in the order and the image interpretation report, and identifies and collects the information such as the order ID and the image ID associated with the tag, but the embodiment is not limited thereto. For example, the collection function 151 may, by analyzing a structured report used for the image interpretation report, identify the content of the medical report. The collection function 151 may be configured, by performing morphological analysis on the content of the image interpretation report, to identify the disease name and the like identified with the finding that is described in the image interpretation report.

The determination function 152 compares the information on the order with the information on the application or the information on the image interpretation report and determines whether a condition is met. The determination function 152 reads out “order ID” and “application ID1” and “application ID2” stored in the report table 143 illustrated in FIG. 5, for example. Then, the determination function 152 refers to the order table 142 and acquires “inspection purpose” of the record that corresponds to “order ID” of the report table 143. Subsequently, the determination function 152 refers to the application table 141 and acquires “inspection purpose” of the record that corresponds to “application ID1” or “application ID2” of the report table 143. Then, the determination function 152 determines whether “inspection purpose” acquired from the order table 142 and “inspection purpose” acquired from the application table 141 match or not. When determined that the two “inspection purposes” do not match, the determination function 152 registers “Y” into “out of purpose” of the relevant record of the report table 143.

For example, “inspection purpose” of the record of the order table 142 corresponding to the order ID “Ord0001” of the record of the report ID “Rep0001” of the report table 143 illustrated in FIG. 5 is, as illustrated in FIG. 4, “tumor identification”. Meanwhile, “inspection purpose” of the record of the application table 141 corresponding to the application ID1 “XXXB” of the record of the report ID “Rep0001” of the report table 143 is, as illustrated in FIG. 3, “blood vessel extraction”. In this case, because the determination function 152 determines that the two “inspection purposes” do not match, the determination function 152 registers “Y” into “out of purpose” of the record of the report ID “Rep0001” of the report table 143. On the other hand, for example, “inspection purpose” of the record of the order table 142 corresponding to the order ID “Ord0003” of the record of the report ID “Rep0003” of the report table 143 illustrated in FIG. 5 is, as illustrated in FIG. 4, “tumor identification”. Meanwhile, “inspection purpose” of the record of the application table 141 corresponding to the application ID1 “XXXA” of the record of the report ID “Rep0003” of the report table 143 is, as illustrated in FIG. 3, “tumor identification”. In this case, because the determination function 152 determines that the two “inspection purposes” match, the determination function 152 registers nothing into “out of purpose” of the record of the report ID “Rep0003” of the report table 143.

The determination function 152 reads out “order ID” and “finding” stored in the report table 143 illustrated in FIG. 5, for example. Subsequently, the determination function 152 refers to the order table 142 and acquires “assumed disease name” of the record that corresponds to “order ID” of the report table 143. Then, the determination function 152 determines whether “assumed disease name” acquired from the order table 142 and “finding” acquired from the report table 143 match or not. When determined that “assumed disease name” and “finding” do not match, the determination function 152 registers “Y” into “contribution” of the relevant record of the report table 143, since it is conceivable that “finding” was changed by the image interpretation of the medical image after image analysis, for example.

For example, the finding “lung cancer” of the record of the report ID “Rep0001” of the report table 143 illustrated in FIG. 5 matches the assumed disease name “lung cancer” of the record of the order table 142 corresponding to the order ID “Ord0001” of the relevant record. In this case, because the determination function 152 determines that “assumed disease name” and “finding” match, the determination function 152 registers nothing into “contribution” of the record of the report ID “Rep0001” of the report table 143. Meanwhile, the finding “lien accessorius” of the record of the report ID “Rep0003” of the report table 143 does not match the assumed disease name “pancreatic masses” of the record of the order table 142 corresponding to the order ID “Ord0003” of the relevant record. In this case, because the determination function 152 determines that “assumed disease name” and “finding” do not match, the determination function 152 registers “Y” into “contribution” of the record of the report ID “Rep0003” of the report table 143.

Next, the output function 153 generates and outputs information about the utilization status of the clinical application in generating the image interpretation report, by using the information stored in the report table 143 on the clinical application utilized for the image analysis of the medical image. At that time, the output function 153 narrows down or sorts the utilization status of clinical applications by various conditions such as the date on which the clinical application was utilized, target portion of the image interpretation report, inspection purpose, and the like. The output function 153 further graphs, for example, the utilization status of the clinical application that was narrowed down or sorted by the various conditions, and outputs the graphed utilization status to a screen and the like of a terminal (not depicted) of the manager of the result management device 10.

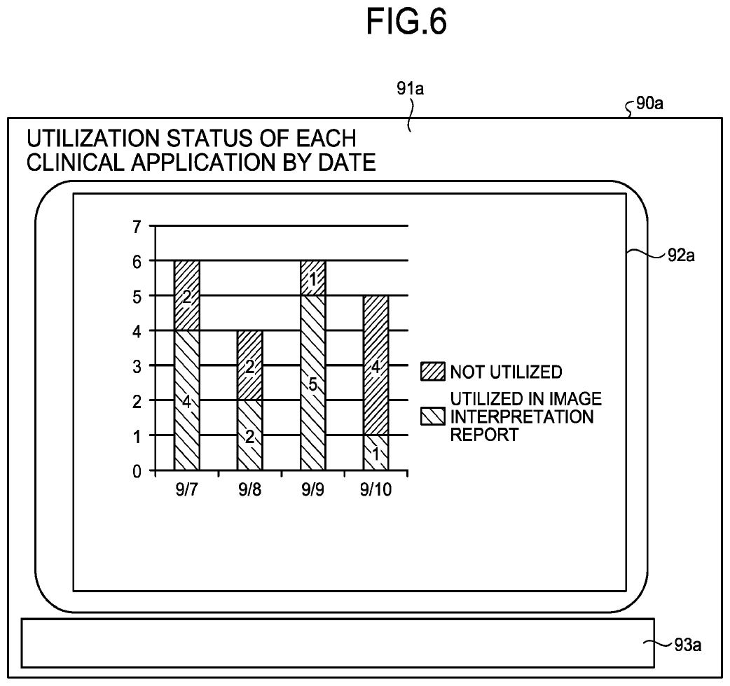

For example, the output function 153 outputs such a graph illustrated in FIG. 6 by sorting, out of the report table 143 illustrated in FIG. 5, the utilization status of clinical applications according to “generation date”. FIG. 6 is a diagram illustrating one example of a result display screen by date in the first embodiment. In a screen 90a illustrated in FIG. 6, a title 91a of the screen, a graph 92a of utilization status of the clinical application by date, and a message field 93a are displayed. As illustrated in FIG. 6, the title 91a of the screen is “utilization status of each clinical application by date”. Furthermore, the graph 92a indicates that “six (6) cases” of image interpretation reports were generated on “September 7” and that, out of the “six (6) cases”, the clinical application was utilized in “four (4) cases” of the image interpretation reports and was not utilized in “two (2) cases” of the image interpretation reports. Similarly, the graph 92a indicates that the clinical application was utilized in “two (2) cases” of the image interpretation reports and was not utilized in “two (2) cases” of the image interpretation reports on “September 8”. Moreover, the graph 92a indicates that the clinical application was utilized in “five (5) cases” of the image interpretation reports and was not utilized in “one (1) case” of the image interpretation report on “September 9” and that the clinical application was utilized in “one (1) case” of the image interpretation report and was not utilized in “four (4) cases” of the image interpretation reports on “September 10”. As illustrated in FIG. 6, in the message field 93a, no message is displayed.

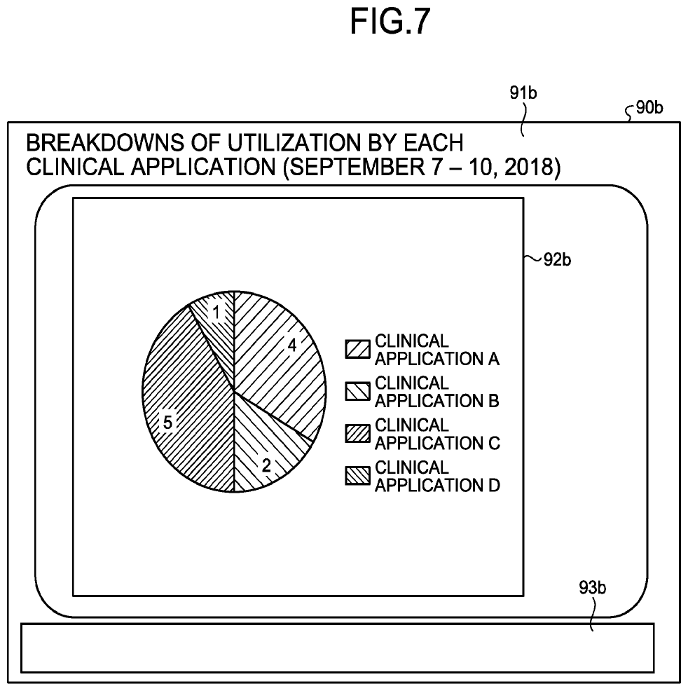

Furthermore, for example, the output function 153 may, out of the report table 143 illustrated in FIG. 5, narrow down the records in which “application ID1” or “application ID2” is registered, that is, the records for which the clinical application was utilized for generation of the image interpretation reports. In this case, the output function 153 may output such a graph illustrated in FIG. 7, by sorting the narrowed down records by “application ID1” or “application ID2”. FIG. 7 is a diagram illustrating one example of a result display screen by each clinical application in the first embodiment. In a screen 90b illustrated in FIG. 7, a title 91b of the screen, a graph 92b of the utilization status by the type of clinical application, and a message field 93b are displayed. As illustrated in FIG. 7, the title 91b of the screen indicates that the graph 92b is a graph presenting “breakdowns of utilization by each clinical application” during the period of Sep. 7 to 10, 2018. The graph 92b indicates that, during the relevant period, “clinical application A” was utilized in “four (4) cases” of the image interpretation reports. Similarly, the graph 92b indicates that, during the relevant period, “clinical application B” was utilized in “two (2) cases” of the image interpretation reports, “clinical application C” was utilized in “five (5) cases” of the image interpretation reports, and “clinical application D” was utilized in “one (1) case” of the image interpretation report. As illustrated in FIG. 7, in the message field 93b, no message is displayed.

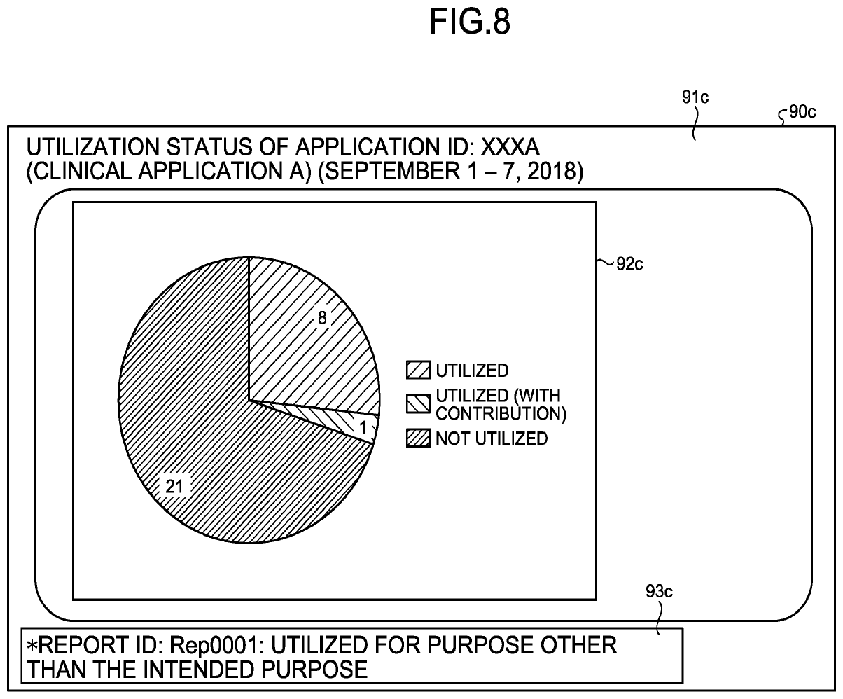

Furthermore, for example, the output function 153 may, out of the report table 143 illustrated in FIG. 5, sort the records according to whether “XXXA” is registered in “application ID1” or “application ID2”. In this case, the output function 153 may output such a graph illustrated in FIG. 8, by further sorting the sorted records by “out of purpose” and “contribution”. FIG. 8 is a diagram illustrating one example of a result display screen of an individual clinical application in the first embodiment. In a screen 90c illustrated in FIG. 8, a title 91c of the screen, a graph 92c of the utilization status of the clinical application A, and a message field 93c are displayed. As illustrated in FIG. 8, the title 91c of the screen indicates that the graph 92c is a graph presenting “utilization status of clinical application A” during the period of Sep. 1 to 7, 2018, and that the application ID of the clinical application A is “XXXA”. The graph 92c indicates that, during the relevant period, “clinical application A” was utilized in “nine (9) cases” of the image interpretation reports and that, out of the “nine (9) cases”, “one (1) case” contributed to the finding. Similarly, the graph 92c indicates that, during the relevant period, “clinical application A” was not utilized in “twenty-one (21) cases” of the image interpretation reports. As illustrated in FIG. 8, in the message field 93c, a message indicating that the clinical application A was utilized for a purpose other than the intended purpose in generating the image interpretation report of the report ID “Rep0001” is displayed.

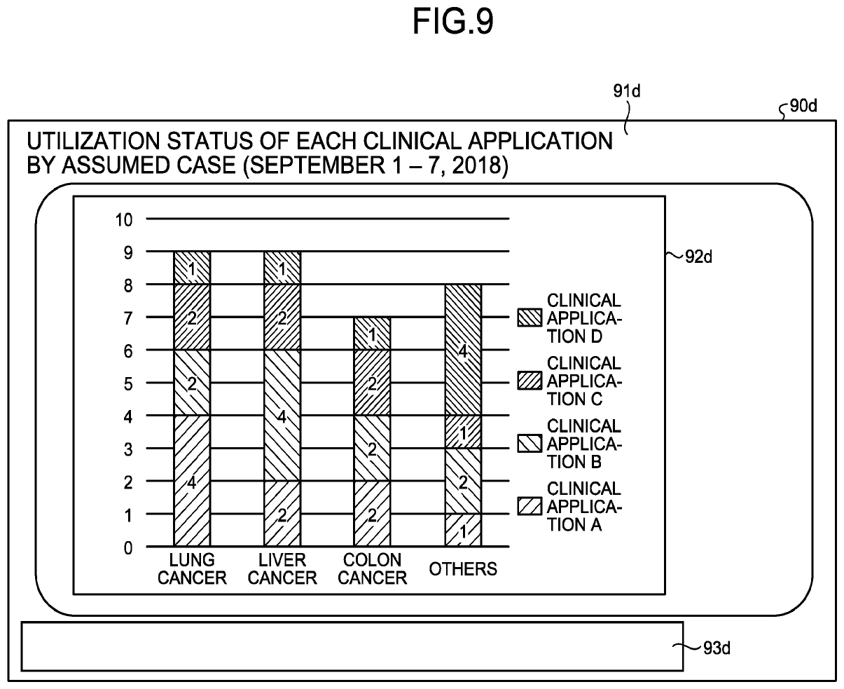

Furthermore, for example, out of the report table 143 illustrated in FIG. 5, the output function 153 may, by narrowing down the records in which “application ID1” or “application ID2” is registered and by further sorting the records according to “assumed disease name” registered in the order table 142, output such a graph illustrated in FIG. 9. FIG. 9 is a diagram illustrating one example of a result display screen by each assumed disease name in the first embodiment. In a screen 90d illustrated in FIG. 9, a title 91d of the screen, a graph 92d of the utilization status of the clinical application by each assumed disease name, and a message field 93d are displayed. As illustrated in FIG. 9, the title 91d of the screen indicates that the graph 92d represents a graph presenting “utilization status of each clinical application by each assumed disease name” during the period of Sep. 1 to 7, 2018. The graph 92d indicates that, with respect to the orders whose assumed disease name are “lung cancer”, during the relevant period, “clinical application A” was utilized in “four (4) cases” of the image interpretation reports, “clinical application B” and “clinical application C” were each utilized in “two (2) cases” of the image interpretation reports, and “clinical application D” was utilized in “one (1) case” of the image interpretation report. Similarly, the graph 92d indicates that, with respect to the orders whose assumed disease name are “liver cancer”, during the relevant period, “clinical application A” and “clinical application C” were each utilized in “two (2) cases” of the image interpretation reports, “clinical application B” was utilized in “four (4) cases” of the image interpretation reports, and “clinical application D” was utilized in “one (1) case” of the image interpretation report. Similarly, the graph 92d indicates that, with respect to the orders whose assumed disease name is “colon cancer”, during the relevant period, “clinical application A”, “clinical application B”, and “clinical application C” were each utilized in “two (2) cases” of the image interpretation reports and “clinical application D” was utilized in “one (1) case” of the image interpretation report. Similarly, the graph 92d indicates that, with respect to the orders whose assumed disease name is “others”, during the relevant period, “clinical application A” and “clinical application C” were each utilized in “one (1) case” of the image interpretation report, “clinical application B” was utilized in “two (2) cases” of the image interpretation reports, and “clinical application D” was utilized in “four (4) cases” of the image interpretation reports. As illustrated in FIG. 9, in the message field 93d, no message is displayed.

The above-described each of various conditions is one example, and the various conditions may be used in combination. For example, the information about the utilization status of clinical applications may be sorted by using a disease name of definite diagnosis as a condition of sorting, and the information about the utilization status of clinical applications may further be narrowed down according to the conditions of the inspection purpose and the assumed disease name.

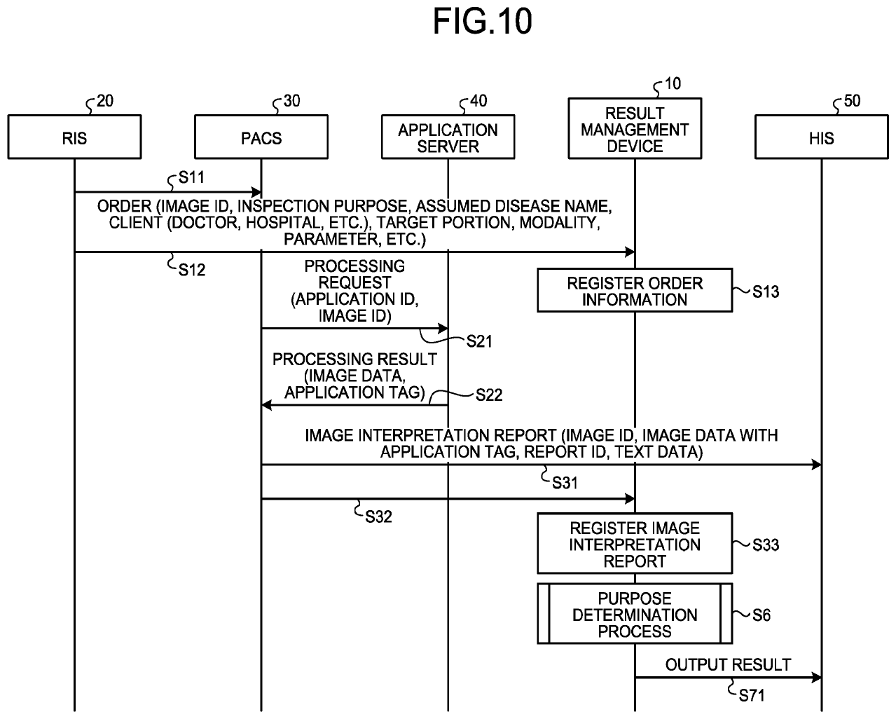

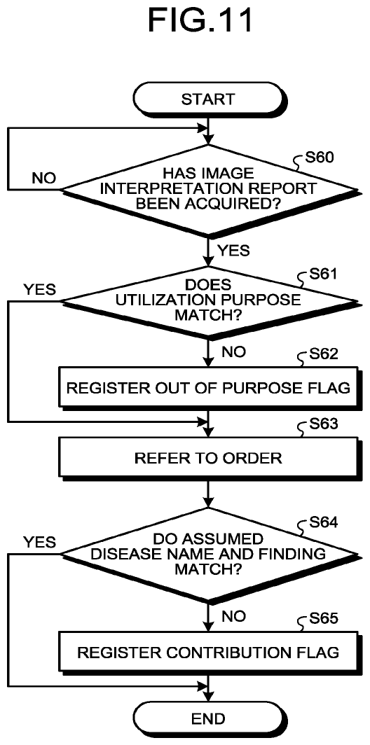

Next, a procedure of processing performed by the result management system 1 in the first embodiment will be described with reference to FIG. 10 and FIG. 11. FIG. 10 is a sequence diagram illustrating one example of a flow of a result management process in the first embodiment.

For example, as illustrated in FIG. 10, in the result management system 1 in the present embodiment, the RIS 20 first outputs an order for generation of an image interpretation report to the PACS 30 via the network NW (Step S11). The order that the RIS 20 outputs includes information such as the image ID, inspection purpose, assumed disease name, doctor or requesting department of hospital and the like, target portion, modality, and parameter and the like, for example. The result management device 10 acquires the information about the order that the RIS 20 output to the PACS 30 (Step S12) and registers the order information into the order table 142 (Step S13).

Subsequently, the PACS 30 outputs to the application server 40 a request for processing of image analysis by a clinical application in generating the image interpretation report (Step S21). The processing request includes the application ID and the image ID, for example. In response to this, the application server 40 outputs to the PACS 30 the data of the DICOM format for which the tag information that uniquely identifies the relevant clinical application was attached to the image data after image analysis (Step S22). Thereafter, the PACS 30 outputs to the HIS 50 the image interpretation report including the image data to which the application tag was attached (Step S31). The image interpretation report that the PACS 30 outputs includes, in addition to the image data to which the application tag was attached, the image ID, report ID, text data in the body of the report, and the like, for example. At this time, the result management device 10 acquires the information about the image interpretation report that the PACS 30 output (Step S32) and registers the information about the image interpretation report into the report table 143 (Step S33).

Then, the result management device 10 executes a purpose determination process (Step S6), and outputs a result of the purpose determination process (S71). FIG. 11 is a flowchart illustrating one example of a purpose determination process in the first embodiment. In FIG. 11, Step S60 is implemented as the processing circuit 15 reads out and executes a program corresponding to the determination function 152 from the memory circuit 14, for example. Each process at Step S61 to Step S65 is implemented as the processing circuit 15 reads out and executes the program corresponding to the determination function 152 from the memory circuit 14, for example.

For example, as illustrated in FIG. 11, in the result management system 1 in the present embodiment, the processing circuit 15 first determines whether the information about the image interpretation report has been acquired from the PACS 30 or not (Step S60). When determined that the information about the image interpretation report has not been acquired (No at Step S60), the processing circuit 15 waits until the information about the image interpretation report is acquired. When determined that the information about the image interpretation report has been acquired (Yes at Step S60), the processing circuit 15 registers the information about the image interpretation report into the report table 143.

Then, the processing circuit 15 determines whether “inspection purpose” of the relevant record registered in the report table 143 matches “inspection purpose” of the relevant clinical application that is corresponding to the application ID of the relevant report and that is registered in the application table 141, that is, whether the utilization purpose of the clinical application matches or not (Step S61). When determined that the utilization purpose of the clinical application matches (Yes at Step S61), the processing circuit 15 moves to Step S63.

Meanwhile, when determined that the utilization purpose of the clinical application does not match (No at Step S61), the processing circuit 15 registers an “out of purpose” flag into the report table 143 (Step S62). Thereafter, processing moves to Step S63.

Subsequently, the processing circuit 15 refers to the order table 142 and identifies “assumed disease name” of the order corresponding to the order ID that is registered in the report table 143 (Step S63). Then, the processing circuit 15 determines whether “assumed disease name” matches “finding” registered in the report table 143 or not (Step S64). When determined that “assumed disease name” matches “finding” (Yes at Step S64), the processing circuit 15 ends the processing.

Meanwhile, when determined that “assumed disease name” does not match “finding” (No at Step S64), the processing circuit 15 registers a “contribution” flag into the report table 143 (Step S65) and ends the processing.

As in the foregoing, because the result management system 1 in the first embodiment is able to grasp the utilization result of the applications utilized for image analysis of medical images easily, it is possible to facilitate identifying how much the clinical applications contribute in generating image interpretation reports or whether the clinical applications are utilized for appropriate purposes, for example.

Modifications

In the above-described embodiment, as one example of a medical report, the configuration in which the image interpretation report is produced by an image interpretation physician has been explained. However, the embodiments are not limited thereto. For example, it may be a configuration in which electronic medical records that are generated in HIS and registered in HIS, the information on patients registered in vendor-neutral archives (VNA), and the like are used as a medical report. For example, by determining whether a diagnosed disease name described in an electronic medical record was changed from the assumed disease name described in an order that was output from the RIS, it is possible to identify whether the clinical application utilized for image analysis of medical image associated with the electronic medical record contributed to identifying the diagnosed disease name.

Furthermore, the configuration in which the utilization result of clinical applications is collected by using the tag information attached to the DICOM images has been explained. However, the embodiments are not limited thereto. For example, when the clinical application outputs, separately from the tag information attached to the DICOM images, information indicating having been used for image analysis, it may be a configuration in which the clinical application utilized for the image analysis is identified by using the information that is output by the clinical application. Furthermore, it may be a configuration in which, by referring to a database that stores therein information about a clinical application that was utilized for image analysis in association with a medical image, the clinical application that was utilized for image analysis of the medical image associated with a medical report is identified. Moreover, by using tag information not used in a medical report but included in a medical image registered in the PACS, the clinical application that was utilized for image analysis of the medical image may be identified. As just described, by acquiring information about the utilization result of the clinical applications from a plurality of sources, it is possible to identify the utilization result of the clinical applications more accurately.

In the above-described embodiment, the configuration in which the utilization status of clinical applications is identified based on the orders by the radiology department acquired from the RIS 20 has been explained. However, the embodiments are not limited thereto. For example, it may be a configuration in which the utilization status of clinical applications is identified based on diagnostic orders of a clinical department acquired from the HIS 50. As a result, also concerning medical reports based on another order of the departments other than the radiology department, it is possible to identify the utilization result of the clinical applications.

The term “processor” used in the description of the above embodiments denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). In this situation, instead of saving the programs in a memory, it is also acceptable to directly incorporate the programs in the circuits of the processors. In that situation, the processors realize the functions by reading and executing the programs incorporated in the circuits thereof. The processors in the present embodiment do not each necessarily have to be structured as a single circuit. It is also acceptable to structure one processor by combining together a plurality of independent circuits so as to realize the functions thereof.

In this situation, the programs executed by the one or more processors are provided as being incorporated, in advance, in a Read-Only Memory, a storage unit, or the like. Alternatively, the programs may be provided as being recorded in a computer-readable storage medium such as a Compact-Disk Read-Only Memory (CD-ROM), a Flexible Disk (FD), a Compact Disk Recordable (CD-R), or a Digital Versatile Disk (DVD), in a file in a format that is installable or executable in those devices. Further, the programs may be provided or distributed by being stored in a computer connected to a network such as the Internet and being downloaded via the network. For example, the programs are each structured with a module including functional units. In actual hardware, as a result of a CPU reading and executing the programs from a storage medium such as a ROM, the modules are loaded into a main storage device and generated in the main storage device.

Further, the configurations of the result management systems in the embodiments are not limited to those described above. For example, another computer such as the PACS 30 or the like may have a part or all of the functions of the processing circuit 15 installed therein or may have a part or all of the content of the storage 14 stored therein. For example, as the PACS 30 implements the entire functions of the processing circuit 15 and stores the entire contents stored in the memory circuit 14, the same configuration as that of the result management device 10 can be implemented on the PACS 30. Further, the functions of the processing circuit 15 included in the management apparatus 10 may be installed in a cloud, and the content of the storage 14 may be stored in a cloud.

According to at least one embodiment in the foregoing, it is possible to easily grasp the utilization result of the applications utilized for image analysis of medical images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A management device comprising a processing circuit configured to:
collect identification information that identifies an application utilized for image analysis of a medical image utilized for generation of a medical report from a plurality of medical reports, and
output information indicating utilization status of the application by using a plurality of pieces of the collected identification information.

2. The management device according to claim 1, wherein the processing circuit collects tag information included in an analyzed image generated by the image analysis, the tag information being capable of identifying an application utilized for the relevant image analysis.

3. The management device according to claim 1, wherein the processing circuit
further determines whether a purpose of capturing the medical image and a utilization purpose of an application utilized for image analysis of the medical image match, and
when determined that the purpose of image capturing does not match the utilization purpose of the application, outputs information indicating that the application was utilized for out of purpose.

4. The management device according to claim 3, wherein the processing circuit
further collects information about an order for capturing the medical image,
determines whether an assumed disease name included in the order and a diagnosed disease name included in the medical report match, and
when the assumed disease name and the diagnosed disease name do not match, outputs information indicating that the application contributed to diagnosis result.

5. A management system comprising a processing circuit configured to collect identification information that identifies an application utilized for image analysis of a medical image utilized for generation of a medical report from a plurality of the medical reports, and to output information indicating utilization status of the application by using a plurality of pieces of the collected identification information.
